(19) United States
(12) Patent Application Publication
(10) Pub. No.: US 2005/0079205 A1
(43) Pub. Date: Apr. 14, 2005

(54) Oleamide-containing composition for animal consumption

(76) Inventor: Cheryl C. Miller, Lawrence, KS (US)

Correspondence Address: HARNESS, DICKEY, & PIERCE, P.L.C, 7700 BONHOMME, STE 400, ST. LOUIS, MO, 63105, US

(21) Appl. No.: 10/938,050

(22) Filed: Sep. 10, 2004

Related U.S. Application Data
(60) Provisional application No. 60/501,688, filed on Sep. 10, 2003.

(51) Int. Cl. A23K 1/165; A61K 31/16

(52) U.S. Cl. 424/442; 514/626

(57) ABSTRACT

This invention is directed generally to compositions (including foods, supplements, treats, toys, etc.) for animal consumption, particularly compositions that tend to aid in weight loss or reduction in weight gain, and particularly compositions that comprise oleamide. This invention also is directed generally to methods for using such compositions. This invention is further directed generally to processes for making such compositions.

### PRIORITY CLAIM TO RELATED PATENT APPLICATION

This patent claims priority to U.S. Provisional Patent Application Ser. No. 60/501,688 (filed Sep. 10, 2003). The entire text of the above-referenced patent application is incorporated by reference into this patent.

### FIELD OF THE INVENTION

This invention is directed generally to compositions (including foods, supplements, treats, toys, etc.) for animal consumption, particularly compositions that tend to aid in weight loss or reducing weight gain, and particularly compositions that comprise oleamide. This invention also is directed generally to methods for using such compositions. This invention is further directed generally to processes for making such compositions.

### BACKGROUND OF THE INVENTION

Endogenous cannabinoids, which are found in the gastrointestinal (GI) tract, have been reported to decrease gastrointestinal motility. See Izzo, A., et al., “Cannabinoid CB1-receptor mediated regulation of gastrointestinal motility in mice in a model of intestinal inflammation”, Br. J. Pharm., 134:563-570 (2001). This decreased gastrointestinal motility, in turn, tends to reduce hunger. Fatty acid amide hydrolase (FAAH), which is present in intestinal epithelial cells, plays a role in controlling gut motility by altering levels of endogenous gastrointestinal anandamide (an endogenous cannabinoid). Specifically, FAAH degrades the endogenous gastrointestinal anandamide, thereby increasing gastrointestinal motility and consequently hunger.

Oleamide (cis-9-octadecenioamide; CH₃(CH₂)₇CH═CH(CH₂)₇CONH₂) is a sleep-inducing fatty acid amide generally produced in the brain of a variety of animals. Like endogenous gastrointestinal anandamide, oleamide is degraded by FAAH. Boger, D., et al., “Exceptionally potent inhibitors of fatty acid amide hydrolase: the enzyme responsible for degradation of endogenous oleamide and anandamide”, Proc. Nat'l Acad. Sci., 9:5044-5049 (2000). Bisogno et al. discuss possible inhibition of FAAH activity through substrate competition involving various FAAH substrates, including oleamide and anandamide. Bisogno, T., et al., “Fatty Acid Amide Hydrolase, an enzyme with many bioactive substrates. Possible therapeutic implications”, Current Pharm. Design, 8(3):125-133 (2002). See also, Bisogno, T., et al., “Fatty acid amide hydrolase, an enzyme with many bioactive substrates. Possible Therapeutic Implications”, Current Pharm. Design, 8(7):533-547 (2002).

DeLuca et al. discuss effects that they reportedly observed when supplementing feed of lactating Jersey cows with up to 3.5% oleamide. The reported effects include a reduction in dry matter intake. See DeLuca, D., et al., “Feeding oleamide to lactating Jersey cows. 2. Effects on nutrient digestibility, plasma fatty acids, and hormones.” J Dairy Sci., 83:569-576 (2000).

Jenkins et al. discuss effects that they reportedly observed when exposing Holstein cows to 4.2% oleamide either through feed or by administering it through a ruminal cannula. The reported effects include a reduction in dry matter intake. In this study, it was concluded that the intake reduction was primarily physiological, rather than due to a unique taste or odor of the oleamide perceived as undesirable by the cows. See Jenkins, T., et al, “Site administration and duration of feeding oleamide to cattle on feed intake and ruminal fatty acid concentrations”, J Anim. Sci., 28:2745-2753 (2000).

Despite years of studies and developments relating to weight loss, there continues to be a need for compositions and methods that aid in weight loss or reduction in the rate of weight gain.

### SUMMARY OF THE INVENTION

This invention is directed to compositions for animal consumption, particularly compositions that tend to aid in weight loss or reducing the rate of weight gain. It is contemplated that such compositions may be suitable for use with a variety of mammalian and non-mammalian animals.

Briefly, therefore, this invention is directed, in part, to a composition for animal consumption, such as, for example, a food, nutritional supplement, treat, or toy. The composition comprises at least about 0.1% oleamide (based on dry weight of the composition).

In one contemplated embodiment, the composition comprises meat, a meat by-product, a dairy product, or an egg product.

In one contemplated embodiment, the composition comprises from about 2% to about 25% of one or more medium chain triglycerides (based on dry weight of the composition).

In another contemplated embodiment, the composition is intended for consumption by a monogastric animal.

In another such contemplated embodiment, the composition is intended for consumption by a carnivorous animal.

In another contemplated embodiment, the composition is intended for consumption by a omnivorous animal.

In another contemplated embodiment, the composition is intended for consumption by a bird.

This invention also is directed to an animal treat that comprises oleamide.

This invention also is directed to an animal toy that comprises oleamide.

This invention also is directed to processes for preparing such compositions, treats, and toys.

This invention also is directed to methods for using such compositions, treats, and toys to aid in weight loss or reducing weight gain.

Further benefits of Applicants' invention will be apparent to one skilled in the art from reading this patent.

### DETAILED DESCRIPTION OF THE PREFERRED EMBODIMENTS

This detailed description of preferred embodiments is intended only to acquaint others skilled in the art with Applicants' invention, its principles, and its practical application so that others skilled in the art may adapt and apply the invention in its numerous forms, as they may be best suited to the requirements of a particular use. This detailed description and its specific examples, while indicating preferred embodiments of this invention, are intended for purposes of illustration only. This invention, therefore, is not limited to the preferred embodiments described in this specification, and may be variously modified.

It has been found in accordance with this invention that the inclusion of oleamide in animal feed can be useful to aid in weight loss or reducing the rate of weight gain. Without being bound to any particular theory, Applicants believe that this benefit may be the result of, for example, a reduced appetite for the food due to reduced palatability. Applicants also believe that the benefit may alternatively (or at least additionally) be the result of physiological effects (particularly satiety-inducing effects) from the oleamide. Specifically, it is believed that the oleamide may compete with gastrointestinal cannabinoids as an FAAH substrate, thereby reducing the amount of gastrointestinal cannabinoid degradation. This reduction in cannabinoid degradation, in turn, is believed to lead to reduced gastrointestinal motility, which consequently leads to reduced hunger and ultimately reduced food intake.

It is contemplated that the compositions and methods of this invention may be useful for a variety of mammals, including non-human mammals such as non-human primates (e.g., monkeys, chimpanzees, etc.), companion animals (e.g., dogs, cats, equine, etc.), farm animals (e.g., goats, sheep, swine, bovine, etc.), laboratory animals (e.g., mice, rats, etc.), and wild and zoo animals (e.g., wolves, bears, deer, etc.).

In some embodiments of this invention, for example, the animal is a monogastric mammal (i.e., a mammal having a single stomach), such as, for example, a non-human primate, dog, cat, rabbit, horse, or swine.

In other embodiments of this invention, the animal is a carnivorous mammal, i.e., a meat-eating mammal.

In other embodiments of this invention, the animal is an omnivorous mammal, i.e., a mammal that eats both plants and meat.

In other embodiments of this invention, the animal is a companion animal.

In other embodiments of this invention, the animal is a cat.

In other embodiments of this invention, the animal is a dog.

In other embodiments of this invention, the animal is a rabbit.

In other embodiments of this invention, the animal is a swine.

In other embodiments of this invention, the animal is a horse.

It also is contemplated that the compositions and methods of this invention may be useful for a variety of non-mammalian animals. In some embodiments of this invention, for example, the composition is intended for consumption by a bird. Contemplated birds include, for example, companion, farm, zoo, and wild birds (e.g., including, for example, song birds, parrots, ducks, geese, chickens, turkeys, ostriches, etc.).

This invention contemplates a variety of oleamide-containing compositions. Contemplated compositions include, for example, foods, supplements, treats, and toys (typically chewable and consumable toys).

The oleamide preferably is present in the composition in an amount that causes the intended animal to reduce voluntary food intake. In general, the concentration of oleamide in a composition of this invention is at least about 0.1% (or from about 0.2% to about 10%, or from about 1% to about 7%, or from about 2% to about 6%, or from about 1% to about 3%, or from about 3% to about 5%) based on the dry weight of the composition.

Typically, the oleamide and other ingredients of the composition are present at concentrations that do not impart, when combined, an odor or flavor that causes the intended animal to perceive the composition to be unacceptable for consumption. In many instances, a desirable odor and flavor can be achieved using aroma or flavor enhancers.

The oleamide and other ingredients preferably are present at concentrations that are not deleterious to the intended animal's health. Thus, for example, the oleamide and other ingredients preferably are present at concentrations that do not cause undesirable effects on digestion, particularly long term undesirable effects on digestion, such as undesirable effects lasting several days or longer. Undesirable effects on digestion may include, for example, constipation or diarrhea.

The oleamide and other ingredients are preferably present at concentrations that correspond to a safe rate of weight loss. Typically, it is preferred that an animal lose no greater than about 2% of its weight per week.

In some embodiments, the oleamide-containing composition is a food. Although both liquid and solid foods are contemplated, solid foods are typically preferred. Where the food is solid, the oleamide may be coated on the food, incorporated into the food, or both. Contemplated foods include both dry foods or wet foods. The non-oleamide components of the food and their preferred proportions include those listed in Table 1.

TABLE 1

| | Preferred proportion of the |
|---|---|
| | composition (% of dry weight of |
| Component | composition) |
| Carbohydrate | from about 0% to about 50%, |
| (preferably a nitrogen-free or | or from about 5% to about 45% |
| essentially nitrogen-free extract) | |
| Protein | from about 5% to about 70%, |
| | or from about 10% to about 70%, |
| | or from about 10% to about 60% |
| Fat | from about 2% to about 50%, |
| | or from about 5% to about 50%, |
| | or from about 5% to about 40% |
| Dietary fiber | from about 0% to about 40%, |
| | or from about 1% to about 20%, |
| | or from about 1% to about 5.5% |
| Nutritional balancing agents | from about 0% to about 15%, |
| (e.g., vitamins, minerals, and trace | or from about 0% to about 10%, |
| elements) | or from about 2% to about 8% |

In a contemplated embodiment, the composition is a food that comprises the following:
(a) at least about 0.1% (or from about 0.2% to about 10%, or from about 1% to about 7%, or from about 2% to about 6%, or from about 1% to about 3%, or from about 3% to about 5%) oleamide; and
(b) at least one of the following:
   (i) from about 5% to about 70% (or from about 10% to about 70%, or from about 10% to about 60%) protein, and
   (ii) from about 2% to about 50% (or from about 5% to about 50%, or from about 5% to about 40%) fat.
      In such an embodiment, it is contemplated that the composition also may, for example, comprise at least one of the following:
(a) no greater than about 50% (or from about 5% to about 45%) carbohydrate,
(b) no greater than about 40% (or from about 1% to about 20%, or from about 1% to about 5.5%) dietary fiber, and
(c) no greater than about 15% (or no greater than about 10%, or from about 2% to about 8%) of one or more nutritional balancing agents.

In another contemplated embodiment, the composition is a food that comprises the following:
(a) at least about 0.1% (or from about 0.2% to about 10%, or from about 1% to about 7%, or from about 2% to about 6%, or from about 1% to about 3%, or from about 3% to about 5%) oleamide, and
(b) from about 5% to about 70% (or from about 10% to about 70%, or from about 10% to about 60%) protein.

In another contemplated embodiment, the composition is a food that comprises the following:
(a) at least about 0.1% (or from about 0.2% to about 10%, or from about 1% to about 7%, or from about 2% to about 6%, or from about 1% to about 3%, or from about 3% to about 5%) oleamide, and
(b) from about 2% to about 50% (or from about 5% to about 50%, or from about 5% to about 40%) fat.

In another contemplated embodiment, the composition is a food that comprises the following:
(a) at least about 0.1% (or from about 0.2% to about 10%, or from about 1% to about 7%, or from about 2% to about 6%, or from about 1% to about 3%, or from about 3% to about 5%) oleamide,
(b) from about 5% to about 70% (or from about 10% to about 70%, or from about 10% to about 60%) protein, and
(c) from about 2% to about 50% (or from about 5% to about 50%, or from about 5% to about 40%) fat.

In another contemplated embodiment, the composition is a food that comprises the following:
(a) at least about 0.1% (or from about 0.2% to about 10%, or from about 1% to about 7%, or from about 2% to about 6%, or from about 1% to about 3%, or from about 3% to about 5%) oleamide,
(b) from about 5% to about 70% (or from about 10% to about 70%, or from about 10% to about 60%) protein,
(c) from about 2% to about 50% (or from about 5% to about 50%, or from about 5% to about 40%) fat,
(d) no greater than about 50% (or from about 5% to about 45%) carbohydrate,
(e) no greater than about 40% (or from about 1% to about 20%, or from about 1% to about 5.5%) dietary fiber, and
(f) no greater than about 15% (or no greater than about 10%, or from about 2% to about 8%) of one or more nutritional balancing agents.

Specific preferred amounts for each component in a composition will depend on a variety of factors including, for example, the species of animal consuming the composition; the particular components included in the composition; the age, weight, general health, sex, and diet of the animal; the animal's consumption rate; the type of composition condition(s) being treated; and the like. Thus, the component amounts may vary widely, and may even deviate from the preferred proportions set forth in this patent.

It is contemplated that the protein in the compositions of the present invention may be supplied by a variety sources, including, plant sources, animals sources, or both. Animal sources include, for example, meat, meat by-products, diary, eggs, etc. Meats include, for example, the flesh of poultry; fish; and mammals (e.g., cattle, swine, sheep, goats, and the like). Meat by-products include, for example, lungs, kidneys, brain, livers, and stomachs and intestines (preferably freed of essentially all or all their contents).

In some contemplated embodiments, the protein comprises meat, a meat by-product, a dairy product, or an egg product. In some such embodiments, for example, the total concentration of meat(s), meat by-product(s), dairy product(s), and egg product(s) in the composition is from about 5% to about 70% (or from about 10% to about 70%, or from about 10% to about 60%).

In some contemplated embodiments, the protein comprises meat or a meat by-product. In some such embodiments, for example, the total concentration of meat(s) and meat by-product(s) in the composition is from about 5% to about 70% (or from about 10% to about 70%, or from about 10% to about 60%).

The fat and carbohydrate in the compositions of the present invention may be supplied by a variety of sources, including, for example, meat, meat by-products, other animal or plant protein sources, grains, and mixtures thereof. Grains include, for example, wheat, corn, barley, and rice.

Fiber in the compositions of the present invention may be supplied from a variety of sources, including, for example, vegetable fiber sources such as cellulose, beet pulp, peanut hulls, and soy fiber.

Particularly in instances when the composition is an animal's food, vitamins and minerals preferably are included in amounts required to avoid deficiency and maintain health. These amounts are readily available in the art. The National Research Council (NRC), for example, provides recommended amounts of such ingredients for farm animals. See, e.g., Nutrient Requirements of Swine (10th Rev. Ed., Nat'l Academy Press, Wash. D.C., 1998), Nutrient Requirements of Poultry (9th Rev. Ed., Nat'l Academy Press, Wash. D.C., 1994), Nutrient Requirements of Horses (5th Rev. Ed., Nat'l Academy Press, Wash. D.C., 1989), etc. And the American Feed Control Officials (AAFCO), for example, provides recommended amounts of such ingredients for dogs and cats. See American Feed Control Officials, Incorp., Official publication, pp. 126-140 (2003). Contemplated vitamins generally useful as food additives include, for example, vitamin A, vitamin B1, vitamin B2, vitamin B6, vitamin B12, vitamin C, vitamin D, vitamin E, vitamin H (biotin), vitamin K, folic acid, inositol, niacin, and pantothenic acid. Contemplated minerals and trace elements generally useful as food additives include, for example, calcium, phosphorus, sodium, potassium, magnesium, copper, zinc, chorine, and iron salts.

The compositions of the present invention may further contain additives known in the art. Preferably, such additives are present in amounts that do not impair the purpose and effect provided by the invention. Examples of contemplated additives include, for example, substances that are functionally beneficial to weight management, substances with a stabilizing effect, organoleptic substances, processing aids, substances that enhances palatability, coloring substances, and substances that provide nutritional benefits.

Contemplated substances that may provide a benefit for weight management include, for example, nonfermentable fiber, carnitine, chrominium picolinate, medium chain triglycerides, and the like. In one such embodiment, for example, the composition comprises from about 2% to about 25% (or from about 5% to about 20%, or from about 7% to about 18%, or from about 12% to about 16%) of one or more medium chain triglycerides (MCT) based on dry weight of the composition. As used in this patent, “MCT” is one or more triglycerides containing saturated fatty acid chains of from about 8 to about 12 carbons. These fatty acid chains are often predominantly caprylic acid (8-carbon) and capric acid (10-carbon) chains, with lesser amounts of caproic acid (6-carbon) and lauric acid (12-carbon) chains. Each fatty acid chain in the triglyceride may be identical or different. Sources of MCT include, for example, coconut oil, macadamia oil, palm oil, palm kernel oil, and mixtures of such oils.

Contemplated stabilizing substances include, for example, substances that tend to increase the shelf life of the composition. Potentially suitable examples of such substances include, for example, preservatives, antioxidants, synergists and sequestrants, packaging gases, stabilizers, emulsifiers, thickeners, gelling agents, and humectants. Examples of emulsifiers and/or thickening agents include, for example, gelatin, cellulose ethers, starch, starch esters, starch ethers, and modified starches.

Contemplated additives for coloring, palatability, and nutritional purposes include, for example, colorants; iron oxide, sodium chloride, potassium citrate, potassium chloride, and other edible salts; vitamins; minerals; and flavoring. The amount of such additives in a composition typically is up to 5% (dry basis of the composition).

Supplements include, for example, a feed used with another feed to improve the nutritive balance or performance of the total. Contemplated supplements include compositions that are fed undiluted as a supplement to other feeds, offered free choice with other parts of an animal's ration that are separately available, or diluted and mixed with an animal's regular feed to produce a complete feed. The AAFCO, for example, provides a discussion relating to supplements in the American Feed Control Officials, Incorp. Official Publication, p. 220 (2003). Supplements may be in various forms including, for example, powders, liquids, syrups, pills, encapsulated compositions, etc.

Treats include, for example, compositions that are given to an animal to entice the animal to eat during a non-meal time. Contemplated treats for canines include, for example, dog bones. Treats may be nutritional, wherein the composition comprises one or more nutrients, and may, for example, have a composition as described above for food. Non-nutritional treats encompass any other treats that are non-toxic. The oleamide can be coated onto the treat, incorporated into the treat, or both.

Toys include, for example, chewable toys. Contemplated toys for dogs include, for example, artificial bones. The oleamide can form a coating on the surface of the toy or on the surface of a component of the toy, be incorporated partially or fully throughout the toy, or both. In a contemplated embodiment, the oleamide is orally accessible by the intended user. There a wide range of suitable toys currently marketed. See, e.g., U.S. Pat. No. 5,339,771 (and references disclosed in U.S. Pat. No. 5,339,771). See also, e.g., U.S. Pat. No. 5,419,283 (and references disclosed in U.S. Pat. No. 5,419,283). It should be recognized that this invention contemplates both partially consumable toys (e.g., toys comprising plastic components) and fully consumable toys (e.g., rawhides and various artificial bones). It should be further recognized that this invention contemplates toys for both human and non-human use, particularly for companion, farm, and zoo animal use, and particularly for dog, cat, or bird use.

In preparing a composition of the present invention, the components of the composition are adjusted so that the oleamide is present in the composition at a concentration of at least about 0.1% (or from about 0.2% to about 10%, or from about 1% to about 7%, or from about 2% to about 6%, or from about 1% to about 3%, or from about 3% to about 5%) based on the dry content of the composition. The oleamide may, for example, be incorporated into the composition during the processing of the formulation, such as during and/or after mixing of other components of the composition. Distribution of these components into the composition can be accomplished by conventional means.

Compositions of the present invention (particularly foods) can be prepared in a canned or wet form using conventional pet food processes. In one contemplated embodiment, ground animal (e.g., mammal, poultry, and/or fish) proteinaceous tissues are mixed with the other ingredients, including fish oils, cereal grains, other nutritionally balancing ingredients, special purpose additives (e.g., vitamin and mineral mixtures, inorganic salts, cellulose and beet pulp, bulking agents, and the like); and water that sufficient for processing is also added. These ingredients preferably are mixed in a vessel suitable for heating while blending the components. Heating of the mixture may be effected using any suitable manner, such as, for example, by direct steam injection or by using a vessel fitted with a heat exchanger. Following the addition of the last ingredient, the mixture is heated to a temperature range of from about 50° F. to about 212° F. Temperatures outside this range are acceptable, but may be commercially impractical without use of other processing aids. When heated to the appropriate temperature, the material will typically be in the form of a thick liquid. The thick liquid is filled into cans. A lid is applied, and the container is hermetically sealed. The sealed can is then placed into conventional equipment designed to sterilize the contents. This is usually accomplished by heating to temperatures of greater than about 230° F. for an appropriate time, which is dependent on, for example, the temperature used and the composition.

Compositions of the present invention (particularly foods) can be prepared in a dry form using conventional processes. In one contemplated embodiment, dry ingredients, including, for example, animal protein sources, plant protein sources, grains, etc., are ground and mixed together. Moist or liquid ingredients, including fats, oils, animal protein sources, water, etc., are then added to and mixed with the dry mix. The mixture is then processed into kibbles or similar dry pieces. Kibble is often formed using an extrusion process in which the mixture of dry and wet ingredients is subjected to mechanical work at a high pressure and temperature, and forced through small openings and cut off into kibble by a rotating knife. The wet kibble is then dried and optionally coated with one or more topical coatings which may include, for example, flavors, fats, oils, powders, and the like. Kibble also can be made from the dough using a baking process, rather than extrusion, wherein the dough is placed into a mold before dry-heat processing.

Treats of the present invention can be prepared by, for example, an extrusion or baking process similar to those described above for dry food. Other processes also may be used to either coat oleamide on the exterior of existing treat forms, or inject it into an existing treat form.

Animal toys of the present invention are typically prepared by coating any existing toy with oleamide.

### EXAMPLES

The following examples are merely illustrative, and not limiting to this disclosure in any way.

### Example 1

Beagles were fed either a dry control food or a dry test food ad libitum for 4 consecutive weeks. The dogs were weighed weekly, and their food intake was recorded daily. The ingredients in the control food and test food are described in Table 2 below:

TABLE 2
Compositions of Control and Test Foods

| | % by Weight | % by Weight |
|---|---|---|
| Ingredient | Control Food | Test Food |
| Rice | 45-55 | 45-55 |
| Poultry By-Product Meal | 20-30 | 20-30 |
| Corn starch | 5-15 | 5-15 |
| Soybean oil | 1-5 | 1-5 |
| Choice white grease | 4 | 0 |
| Oleamide | 0 | 4 |
| Inorganic Salts (calcium carbonate, salt, | 1-3 | 1-3 |
| dicalcium phosphate, potassium chloride) | | |
| Vitamins | 0.01-0.2 | 0.01-0.2 |
| Minerals | 0.01-0.2 | 0.01-0.2 |

The results of this experiment are shown below in Table 3 and Table 4. As shown in Table 3, a reduction in voluntary food intake was observed over the course of the study for the dogs fed the 4% oleamide test food relative to the dogs that were fed the control food. And, as shown in Table 4, a greater loss in weight was observed over the course of the study for the dogs fed the 4% oleamide test food relative to the dogs that were fed the control food.

TABLE 3
Food Consumption (grams consumed/SEM)

| Treat- | | | | | |
|---|---|---|---|---|---|
| ment | Week 1 | Week 2 | Week 3 | Week 4 | Total |
| Dogs | 210 ± 14.2 | 221.6 ± 11 | 211.7 ± 11.8 | 216.2 ± | 215 ± 4 |
| fed | | | | 14.5 | |
| test | | | | | |
| food | | | | | |
| Dogs | 277.8 ± 13.2 | 249.4 ± 8.7 | 267.5 ± 16.4 | 268.2 ± | 266 ± 5 |
| fed | | | | 18.4 | |
| control | | | | | |
| food | | | | | |

TABLE 4
Mean Weekly Body Weight Change (kg)

| Treatment | Week 1 | Week 2 | Week 3 | Week 4 | Total |
|---|---|---|---|---|---|
| Dogs fed test | −0.007 | −0.225 | −0.281 | −0.089 | −0.603 |
| food | | | | | |
| Dogs fed | 0.561 | −0.150 | −0.260 | 0.012 | 0.161 |
| control food | | | | | |

### Example 2

Beagles with body condition scores of>4 (1=lean, and 5=obese) were fed either a control food or test food ad libitum for 63 days. In both studies, the dogs were weighed weekly, and their food intake was recorded daily. The ingredients in the control food and test food are as described in Table 2 above.

The results of this experiment are shown below in Table 5 and Table 6. As shown in Table 5, a reduction in voluntary food intake was observed over the course of the study for the dogs fed the 4% oleamide test food relative to the dogs that were fed the control food. And, as shown in Table 6, a greater loss in weight was observed over the course of the study for the dogs fed the 4% oleamide test food relative to the dogs that were fed the control food.

TABLE 5
Food Consumption (grams consumed/SEM)

| Treatment | Week 1 | Week 2 | Week 3 | Week 4 | Week 5 | Week 6 | Total |
|---|---|---|---|---|---|---|---|
| Dogs fed test | 141 ± 27 | 172 ± 19 | 198 ± 18 | 202 ± 12 | 198 ± 20 | 195 ± 18 | 184 ± 21 |
| food | | | | | | | |
| Dogs fed | 167 ± 28 | 218 ± 19 | 254 ± 23 | 273 ± 22 | 255 ± 24 | 264 ± 26 | 239 ± 29 |
| control food | | | | | | | |

TABLE 6
Mean Weekly Body Weight Change (kg)

| Treat- | | | | | | Week | |
|---|---|---|---|---|---|---|---|
| ment | Week 1 | Week 2 | Week 3 | Week 4 | Week 5 | 6 | Total |
| Dogs | −0.89 | −0.24 | 0 | 0.10 | −0.15 | 0.07 | −1.12 |
| fed | | | | | | | |
| test | | | | | | | |
| food | | | | | | | |
| Dogs | −0.62 | 0 | 0.26 | 0.27 | −0.08 | 0.32 | 0.140 |
| fed | | | | | | | |
| control | | | | | | | |
| food | | | | | | | |

The results of Example 1 and Example 2 indicate that addition of oleamide to dog food corresponds to a reduction in food intake and body weight over time. The reduction in food intake was voluntary, and the loss of weight occurred at a safe and desirable rate (i.e., not exceeding 2% per week).

### Example 3

Two palatability assessment protocols were used to assess the palatability of a food containing oleamide. The first protocol was a standard two-pan test, which used total food consumed to assess palatability. The second protocol was a cognitive palatability assessment (CPA) protocol, in which selective responses to one of three objects was used to determine food preferences. See Araujo, J., et al., “A novel cognitive palatability assessment protocol for dogs”, J Anim. Sci., 82:2200-2208 (2004). This latter procedure is particularly useful for assessing palatability of foods with satiety-inducing properties, for which the standard two-pan test would be biased in favor of a food without such properties.

The following three test foods were used in this experiment:
1. Control Food: Hill's Prescription Diet Canine w/d food
2. Low-Dose Food: Hill's Prescription Diet Canine w/d food supplemented with 2% oleamide.
3. High-Dose Food: Hill's Prescription Diet Canine w/d food supplemented with 4% oleamide.
   The dogs were fed once daily for one hour.

The two-pan-test protocol indicated a preference for the control food over the high-dose oleamide food, and a smaller preference for the control food over the low-dose oleamide food. The differences in preferences, however, lacked statistical significance. This absence of significance may be due to the small sample size and individual variability. The data also varied as a function of food experience.

Dogs tested using the CPA protocol had higher total intake and a greater preference for the control food than did the dogs tested first on the two-pan protocol. The CPA protocol revealed a statistically significant preference for the control food over the high-dose food, and a non-significant preference for the low-dose food over the control food. The results of the CPA protocol indicated stable individual preferences. The absence of significance may be a reflection of individual differences in variability.

The dogs consumed all the control food and low-dose food provided. By contrast, only 25% of the dogs consumed all the high-dose food provided.

### Example 4

Young beagle dogs were fed one of the following foods.
1. Control Food: Hill's Prescription Diet Canine w/d food
2. Low-Dose Food: Hill's Prescription Diet Canine w/d food supplemented with 2% oleamide.
3. High-Dose Food: Hill's Prescription Diet Canine w/d food supplemented with 4% oleamide.
   The dogs were fed once daily for one hour. Activity was assessed using an actiwatch placed within a specially designed collar. Continuous activity data was recorded for 7 consecutive days and nights for each food.

This experiment revealed significantly greater daytime activity in the dogs that were fed the high-dose diet. There was no significant differences in daytime activity in dogs fed the control or low-dose diets. And there was no significant differences in nighttime activity for any of the dogs.

All the references cited above are incorporated by reference into this patent.

The words “comprise”, “comprises”, and “comprising” are to be interpreted inclusively rather than exclusively.

The above detailed description of preferred embodiments is intended only to acquaint others skilled in the art with the invention, its principles, and its practical application so that others skilled in the art may adapt and apply the invention in its numerous forms, as they may be best suited to the requirements of a particular use. This invention, therefore, is not limited to the above embodiments, and may be variously modified.

1. A composition for animal consumption, wherein the composition comprises:
at least about 0.1% oleamide (based on dry weight of the composition); and
at least one ingredient selected from the group consisting of meat, a meat product, a dairy product, and an egg product.

2. A composition according to claim 1, wherein the composition comprises from about 0.2% to about 10% oleamide (based on dry weight of the composition).

3. A composition according to claim 2, wherein the composition comprises from about 1% to about 3% oleamide (based on dry weight of the composition).

4. A composition according to claim 2, wherein the composition comprises from about 3% to about 5% oleamide (based on dry weight of the composition).

5. A composition according to claim 2, wherein:
the composition comprises at least one ingredient selected from the group consisting of meat and a meat product, and
the total concentration of meat and meat product in the composition is from about 5% to about 70% (based on dry weight of the composition).

6. A composition according to claim 2, wherein the composition comprises food manufactured for consumption by an animal selected from the group consisting of a dog and cat.

7. A composition for animal consumption, wherein the composition comprises:
at least about 0.1% oleamide (based on dry weight of the composition); and
from about 2% to about 25% of one or more medium chain triglycerides (based on dry weight of the composition).

8. A composition according to claim 7, wherein the composition comprises from about 2% to about 6% oleamide (based on dry weight of the composition).

9. A composition for animal consumption, wherein:
the composition comprises at least about 0.1% oleamide (based on dry weight of the composition); and
the composition is manufactured for consumption by an animal selected from the group consisting of a monogastric animal, omnivorous animal, carnivorous animal, and bird.

10. A composition according to claim 9, wherein the composition comprises from about 2% to about 6% oleamide (based on dry weight of the composition).

11. A composition according to claim 10, wherein the composition is manufactured for consumption by a monogastric animal.

12. A composition according to claim 11, wherein the composition is manufactured for consumption by an animal selected from the group consisting of a rabbit, cat, dog, horse, and swine.

13. A composition according to claim 10, wherein the composition is manufactured for consumption by an animal selected from the group consisting of an omnivore and carnivore.

14. A process for making a composition of claim 1, wherein the process comprises incorporating oleamide into a food.

15. A process for making a composition of claim 7, wherein the process comprises incorporating oleamide into a food.

16. A process for making a composition of claim 9, wherein the process comprises incorporating oleamide into a food.

17. A method for aiding an animal in losing weight or reducing weight gain, wherein the method comprises feeding the animal a composition of claim 1.

18. A method for aiding an animal in losing weight or reducing weight gain, wherein the method comprises feeding the animal a composition of claim 7.

19. A method for aiding an animal in losing weight or reducing weight gain, wherein the method comprises feeding the animal a composition of claim 9.

20. A method according to claim 19, wherein the animal is selected from the group consisting of a cat and dog.

21. An animal treat, wherein the treat comprises oleamide.

22. An animal treat according to claim 21, wherein:
the treat comprises from about 2% to about 6% oleamide (based on dry weight of the treat), and
the treat is manufactured for consumption by an animal selected from the group consisting of a cat and dog.

23. An animal toy, wherein the toy comprises oleamide.

24. An animal toy according to claim 23, wherein the toy is manufactured for use by an animal selected from the group consisting of a cat and dog.
